(19) United States
(12) Patent Application Publication
(10) Pub. No.: US 2025/0281467 A1
(43) Pub. Date: Sep. 11, 2025

(54) MODULATORS OF INTRACELLULAR CHLORIDE CONCENTRATION FOR USE IN THE TREATMENT OF COGNITIVE DECLINE

(71) Applicants: FONDAZIONE ISTITUTO ITALIANO DI TECNOLOGIA, Genova (IT); FONDAZIONE TELETHON ETS, Roma (IT)

(72) Inventors: Laura Cancedda, Genova (IT); Annalisa Savardi, Genova (IT); Marco De Vivo, Genova (IT); Marco Borgogno, Genova (IT)

(21) Appl. No.: 18/858,015

(22) PCT Filed: Apr. 21, 2023
(86) PCT No.: PCT/IB2023/054081

§ 371 Date: Oct. 18, 2024

(30) Foreign Application Priority Data
Apr. 22, 2022 (IT) 102022000008078

(51) Int. Cl. A61K 31/437; A61K 31/401; A61K 31/4025; A61K 31/404; A61K 31/422; A61K 31/4439; A61P 25/28

(52) U.S. Cl. CPC A61K 31/437; A61K 31/401; A61K 31/4025; A61K 31/404; A61K 31/422; A61K 31/4439; A61P 25/28

(57) ABSTRACT

The present invention relates to KCC2 inhibitors for the use in the treatment of cognitive decline, preferably age-related cognitive decline. In particular, compounds of formula (III) or compounds 65-69 are provided.

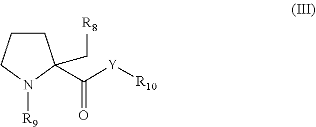

### CROSS-REFERENCE TO RELATED APPLICATIONS

This patent application claims priority from Italian Patent Application No. 102022000008078 filed on Apr. 22, 2022, the entire disclosure of which incorporated is herein by reference.

### TECHNICAL FIELD

The present invention relates to KCC2 inhibitors for the use in the treatment of cognitive decline, for example age-related cognitive decline.

### BACKGROUND

Aging is a physiological process that leads to the occurrence of a series of conditions, at times also severe, that considerably influence the quality of life of elderly people as well as that of their families.

A large number of these disorders affect the central nervous system and can lead to cognitive and memory defects, dementia, sleep disorders, increased susceptibility to convulsions and adverse reactions to benzodiazepine based drugs. In particular, cognitive decline is a common event that might occur with aging of the central nervous system, even in the absence of a diagnosis of neurodegenerative disease (e.g. Alzheimer's, Parkinson's, Huntington's). This condition is characterized by memory, language, thought and judgment problems with various degrees of severity. More severe cases can influence the autonomy and daily activities of elderly people.

Currently, no specific pharmacological treatments are available to mitigate cognitive decline, above all age-related cognitive decline. To try to preserve intellectual performance in aging people, it is possible to act in advance attempting to reduce a series of risk factors, such as obesity, hypertension, hypercholesterolemia, diabetes, smoking, alcohol abuse and an excessively stressful lifestyle. This can be obtained through a healthy lifestyle (balanced diet, rich in fruit and vegetables, fish, whole grain cereals, vegetable oils and nuts, regular physical activity, low alcohol consumption, no smoking, weight control) and targeted drug therapies (above all in the case of hypertension, hypercholesterolemia and diabetes). Other valid preventive strategies consist in keeping mentally active (reading, visiting exhibitions, cinemas and theatres, using technological devices, etc.) and engaging in interactive activities at family and social level (looking after grandchildren, taking part in voluntary work activities, following courses of any type, organizing parties and group travel, etc.). These activities can favour brain plasticity, which is fundamental for cognitive functions.

Therefore, in the art there is the need for new compounds capable of mitigating cognitive decline.

### SUMMARY

Consequently, the object of the present invention is to provide compounds capable of mitigating cognitive decline, preferably age-related cognitive decline.

This object is achieved thanks to the use of KCC2 inhibitors of claim 1.

Among the main regulators of brain plasticity (and hence of learning and memory) is the neurotransmitter GABA. GABA is the main inhibitory neurotransmitter of the central nervous system (CNS) and regulates neuron excitability. GABA exerts its action by binding to different GABA receptors. These include the ionotropic GABA_{A} receptor (GABA_{A}R). GABA_{A}Rs are ligand-gated ion channels that respond to GABA binding by allowing neuron permeability of the chlorine ion (Cl⁻) through the cell membrane. Depending on its concentration gradient across the cell membrane and the membrane resting potential of the neuron, Cl⁻ can flow through the GABA_{A}R in both directions. Therefore, maintaining and regulating the level of concentration of Cl⁻ in neurons is crucial for proper functioning of GABAergic signalling.

The main regulators of Cl⁻ homeostasis in neurons are the NKCC1 cotransporter (Cl⁻ importer) and the KCC2 transporter (Cl⁻ exporter).

During early neurological development, a high concentration of Cl⁻ is present inside the cell owing to a high NKCC1/KCC2 expression ratio (due to low expression of KCC2). Therefore, opening of the GABA_{A}R causes an outflow of Cl⁻ from the cell and therefore GABA activation leads to depolarization of the membrane, and possibly to excitation.

After physiological development of the brain, the adult neurons in the CNS have lower levels of Cl⁻ owing to a lower NKCC1/KCC2 expression ratio (due to an increased expression of KCC2). In this situation, opening of the GABA_{A}R leads to an inflow of Cl⁻, which generates hyperpolarizing transmission, and therefore to an inhibitory impulse.

The inventors have shown that there is a significant increase in the expression of KCC2 in the brains of elderly people and mice.

Therefore, this transporter has proved to be a potential biological target for new therapies.

### BRIEF DESCRIPTION OF THE DRAWINGS

The present invention will now be described in detail with reference to the accompanying drawings, wherein:

FIG. 1 illustrates the evaluation of the expression of KCC2 in the hippocampus of elderly people and mice. a) Quantification of KCC2 in hippocampus samples of elderly individuals (n=6) compared with gender-matched young controls (n=7) (two tailed t-test, ** P<0.01). b) Quantification of KCC2 in hippocampus samples of elderly mice (n=16) compared to young controls (n=13) (two tailed t-test, * P<0.05).

FIG. 2 illustrates the evaluation of the effect of benzodiazepines in elderly mice which is indicative of a potentiated GABAergic transmission. a) Quantification of the immobility time during the open field test of mice treated with vehicle (young, n=7, elderly, n=6) or diazepam (young, n=7, elderly, n=7) (two-way ANOVA, Tukey post hoc test, *** P<0.001). b) Quantification of the distance travelled during the open field test by mice treated with vehicle (young, n=7, elderly, n=6) or diazepam (young, n=7, elderly, n=7) (two-way ANOVA, Tukey post hoc test, ** P<0.01, *** P<0.001).

FIG. 3 illustrates the evaluation of the efficacy of compound 32 (KCC2 inhibitor) to restore impaired memory in elderly mice. a) Quantification of the discrimination index of the NOR test in young (n=13) and elderly (n=10) mice (two tailed t-test, ** P<0.01). b) Quantification of the discrimination index of the NOR test in mice treated with vehicle (young, n=12, elderly, n=9) or compound 32 (young, n=10, elderly, n=6) (two-way ANOVA, Tukey post hoc test, ** P<0.01, ** P<0.001).

### DESCRIPTION OF EMBODIMENTS

Recent results obtained by the inventors indicate that the expression of KCC2 is up-regulated in elderly humans (75-80 years) and elderly mice (>24 months) compared to adults. This alteration leads to a hyper-inhibitory phenotype that results in oversedation following the administration of benzodiazepines and in cognitive decline in animal models.

In particular, it was found that in vivo treatment with different KCC2 inhibitors can reduce age-related cognitive decline and other conditions characterized by altered Cl⁻ homeostasis or by altered GABA_{A}ergic transmission in which KCC2 is overexpressed or over-functioning.

The following paragraphs provide definitions of the various chemical portions of the compounds according to the invention and are intended to be uniformly valid in the whole of the description and in the claims, unless another definition explicitly presented provides a broader definition.

As used herein, the term “alkyl” refers to saturated aliphatic hydrocarbon groups.

Non-limiting examples of alkyl groups according to the invention are, for example, methyl, ethyl, propyl, isopropyl, n-butyl, iso-butyl, tert-butyl, n-pentyl, iso-pentyl, n-hexyl, and the like.

As used herein, the term “cycloalkyl” refers to a saturated or partially unsaturated carbocyclic group having a single ring. It includes cycloalkenyl groups.

Non-limiting examples of cycloalkyl groups according to the invention are, for example, cyclopropane, cyclobutane, cyclopentane, cyclohexane, cycloheptane, cyclopentene, cyclohexene, cyclohexadiene and the like.

As used herein, the term “alcoxyl” refers to an alkyl group that is bonded to the rest of the compound by an oxygen atom.

As used herein, the term “halogen” refers to fluorine, chlorine, bromine and iodine.

The expression “pharmaceutically acceptable salts” refers to salts of the compounds of Formula (III) identified hereunder which maintain desired biological activity and are accepted by the regulatory authorities.

As used herein, the term “salt” refers to any salt of a compound according to the present invention prepared from an inorganic or organic acid or base and to internally formed salts. Typically, these salts have a physiologically acceptable anion or cation.

Moreover, the compounds of Formula (III) can form an acid addition salt or a salt with a base, depending on the type of substituents, and these salts are included in the present invention, as long as they are pharmaceutically acceptable salts.

Examples of these salts include, without limitation, acid addition salts formed with inorganic acids (for example, acid, hydrochloric acid, hydrobromic sulphuric acid, phosphoric acid, nitric acid, and the like), and salts formed with organic acids, such as acetic acid, trifluoroacetic acid, oxalic acid, tartaric acid, succinic acid, malic acid, fumaric acid, maleic acid, ascorbic acid, benzoic acid, alginic acid, polyglutamic acid, methanesulfonic acid, p-toluenesulfonic acid, and naphthalene sulfonic acid.

The compounds of Formula (III) containing acid protons can be converted into their forms of therapeutically active non-toxic base addition salts, for example metal or amine salts, by treatment with suitable organic and inorganic bases. Suitable forms of base salts include, for example, ammonium salts, alkaline and alkaline earth metal salts, for example salts of lithium, sodium, potassium, magnesium, calcium, and the like, salts with organic bases, for example salts of N-methyl-D-glucamine, hydrabamine, and salts with amino acids such as, for example, arginine, lysine, and the like.

Physiologically or pharmaceutically acceptable salts are particularly suitable for medical applications due to their high solubility in water compared to the original compound.

Pharmaceutically acceptable salts can also be prepared from other salts, including other pharmaceutically acceptable salts of the compounds of Formula (III) using conventional methods.

Those skilled in the art of organic chemistry will appreciate that many organic compounds can form complexes with solvents in which they are made to react or from which they are made to precipitate or are crystallized. These complexes are known as “solvates”. For example, a complex with water is known as “hydrate”. The solvates of the compounds of the invention fall within the scope of the invention. The compounds of Formula (III) can be easily isolated together with solvent molecules by crystallization or evaporation of a suitable solvent to provide the corresponding solvates.

The compounds of Formula (III) can be in crystalline form. In some embodiments, the crystalline forms of the compounds of Formula (III) are polymorphic.

The present invention also includes isotopically labelled compounds, which are identical to those presented in Formula (III) and following, but are different due to the fact that one or more atoms are replaced by an atom having a different atomic mass or mass number/or by the atomic mass or by the mass number usually found in nature. Examples of isotopes that can be incorporated in the compounds of the invention and relative pharmaceutically acceptable salts include isotopes of hydrogen, carbon, nitrogen, oxygen, sulphur, fluorine, and chlorine, for example 2H, 3H, 11C, 13C, 14C, 15N, 17O, 18O, 35S, 18F, 36Cl.

The compounds of the present invention and the salts of said compounds that pharmaceutically acceptable contain the aforesaid isotopes and/or other isotopes of other atoms fall within the scope of the present invention. The isotopically labelled compounds of the present invention, for example those in which radioactive isotopes such as 3H, 14C are incorporated, are useful in drug assays and/or substrate tissue distribution assays. Tritium isotopes, i.e., 3H, and carbon-14 isotopes, i.e., 14C, are particularly preferred for their ease of preparation and detectability. The isotopes 11C and 18F are particularly useful in PET (Positron Emission Tomography). Moreover, substitution with heavier isotopes such as deuterium, i.e., 2H, can achieve some therapeutic advantages that occur due to greater metabolic stability, for example an increase in the in vivo half-life or reduced dosage requirements and, therefore, can be preferred in some circumstances. In general, the isotopically labelled compounds of Formula (III) and following of this invention can be prepared by substituting a non-isotopically labelled reagent with an easily available isotopically labelled reagent.

Some groups/substituents included in the present invention can be present as isomers or in one or more tautomeric forms. Consequently, in some embodiments, the compounds of Formula (III) in some cases can exist in the form of other tautomers or geometrical isomers, depending on the type of substituents. In the present specification, although the compounds can be described in only one form of such isomers, the present invention includes all these isomers, isolated forms of the isomers or a mixture thereof. Moreover, in some cases the compounds of Formula (III) can have asymmetric carbon atoms or axial asymmetries and, correspondingly, may exist in the form of optical isomers such as an (R) form, an (S) form, and the like. All such isomers, including racemates, enantiomers and mixtures thereof, are included in the scope of the present invention.

In particular, all stereoisomeric forms, including enantiomers, diastereomers and mixtures thereof, including racemates, are included within the scope of the present invention, and unless otherwise indicated the general reference to the compounds of Formula (III) includes all stereoisomeric forms.

In general, the compounds or salts of the invention should be interpreted as excluding those compounds (if present) which are so chemically unstable, either per se or in water, to be clearly unsuitable for pharmaceutical use through all administration routes, whether oral, parenteral, or of other type. These compounds are known to the skilled chemist.

According to a first aspect of the invention, KCC2 inhibitors for the use in the treatment of cognitive decline, preferably age-related cognitive decline are therefore provided. In particular, according to an embodiment, KCC2 inhibitors have formula (III):

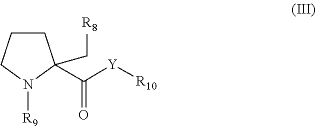

or its pharmaceutically acceptable salts, solvates and tautomers wherein:
Y is selected from the group consisting of O, CH₂ and NR₁₁ where R₁₁ is selected from the group consisting of H and C₁-C₄ alkyl;
R₈ is selected from the group consisting of C₁-C₄ alkyl, phenyl-C₁-C₃ alkyl, C₃-C₆ cycloalkyl, pyridyl, thienyl, and phenyl optionally substituted with at least one substituent selected from the group consisting of C₁-C₃ alkyl, C₁-C₃ alkoxyl, halogen-C₁-C₃ alkyl, halogen, methylsulphonyl, cyano;
R₉ is selected from the group consisting of H, C₁-C₄ alkyl, methylsulphonyl, C₁-C₃ alkyl-NHCO—, C₁-C₄ alkyl-CO—, phenyl-CO—, C₃-C₆ cycloalkyl-CO—, C₁-C₄ alkyl-COO;
R₁₀ is selected from the group consisting of H, C₁-C₄ alkyl, C₃-C₆ cycloalkyl-C₁-C₃ alkyl, pyridyl-C₁-C₃ alkyl, phenyl-C₁-C₃ alkyl, linear or branched, wherein the phenyl group is optionally substituted with a substituent selected from the group consisting of halogen, C₁-C₃ alkyl, halogen-C₁-C₃ alkyl, methylsulphonyl, provided that when Y is NR₁₁, R₁₀ is benzyl. In particular, for the inhibitors of formula (III):
Y is selected from the group consisting of O and NR₁₁ wherein
R₁₁ is selected from the group consisting of H and methyl;
R₈ is selected from the group consisting of methyl, benzyl, cyclohexyl, pyridyl, thienyl and phenyl optionally substituted with at least one group selected from the group consisting of methyl, methoxyl, trifluoromethyl, fluorine, chlorine, methylsulphonyl and cyano;
R₉ is selected from the group consisting of H, ethyl, methylsulphonyl, ethylamide, CH₃—CO—, CH₃CH₂—CO—, phenyl-Co, cyclopropyl-CO, CH₃—COO—;
R₁₀ is selected from the group consisting of H, methyl, benzyl, 1-phenylethyl, 2-phenylethyl, cyclohexyl-CH₂—, pyridyl-CH₂—, chlorophenyl-CH₂—, methylphenyl-CH₂—, trifluorophenyl-CH₂—, methylsulphonylphenyl-CH₂—.

According to a further embodiment, the KCC2 inhibitors are compounds 65, 66, 67, 68, 69 or their pharmaceutically acceptable salts, solvates and tautomers:

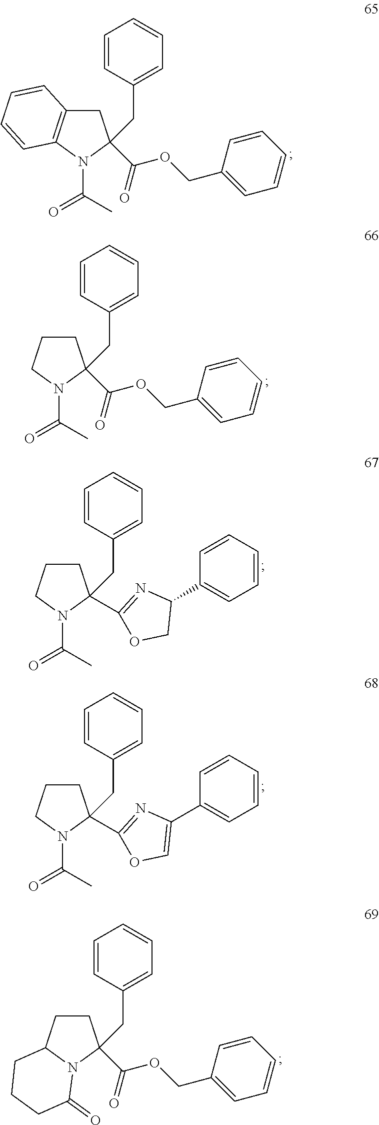

More in particular, the inhibitors of formula (III) are selected from the group consisting of:

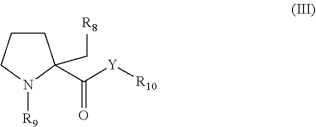

| Com- | | | | |
|---|---|---|---|---|
| pound | Y | R₈ | R₉ | R₁₀ |
| 21 | O | phenyl | COCH₃ | CH₂-phenyl |
| 22 | O | CH₂-phenyl | COCH₃ | CH₂-phenyl |
| 23 | O | CH₃ | COCH₃ | CH₂-phenyl |
| 24 | O | cyclohexyl | COCH₃ | CH₂-phenyl |
| 25 | O | 4-pyridyl | COCH₃ | CH₂-phenyl |
| 26 | O | 3-thienyl | COCH₃ | CH₂-phenyl |
| 27 | O | 4-CH₃-phenyl | COCH₃ | CH₂-phenyl |
| 28 | O | 4-OCH₃-phenyl | COCH₃ | CH₂-phenyl |
| 29 | O | 4-CF₃-phenyl | COCH₃ | CH₂-phenyl |
| 30 | O | 4-F-phenyl | COCH₃ | CH₂-phenyl |
| 31 | O | 4-Cl-phenyl | COCH₃ | CH₂-phenyl |
| 32 | O | 4-(SO₂Me)- | COCH₃ | CH₂-phenyl |
| | | phenyl | | |
| 33 | O | 4-CN-phenyl | COCH₃ | CH₂-phenyl |
| 34 | O | 3-CN-phenyl | COCH₃ | CH₂-phenyl |
| 35 | O | 2-CN-phenyl | COCH₃ | CH₂-phenyl |
| 36 | O | 3-(SO₂Me)- | COCH₃ | CH₂-phenyl |
| | | phenyl | | |
| 37 | O | 3-CF₃-phenyl | COCH₃ | CH₂-phenyl |
| 38 | O | (3-CF₃, 4-F)- | COCH₃ | CH₂-phenyl |
| | | phenyl | | |
| 39 | O | (3-CF₃, 4- | COCH₃ | CH₂-phenyl |
| | | OCH₃)-phenyl | | |
| | | h | | |
| 40 | O | (2-CF₃, 4-F)- | COCH₃ | CH₂-phenyl |
| | | phenyl | | |
| 41 | NH | phenyl | COCH₃ | CH₂-phenyl |
| 42 | NCH₃ | phenyl | COCH₃ | CH₂-phenyl |
| 43 | O | phenyl | COCH₃ | H |
| 44 | O | phenyl | COCH₃ | CH₃ |
| 45 | O | phenyl | COCH₃ | CH(CH₃) |
| | | | | phenyl |
| 46 | O | phenyl | COCH₃ | CH₂CH₂phenyl |
| 47 | O | phenyl | COCH₃ | CH₂cyclohexyl |
| 48 | O | phenyl | COCH₃ | CH₂(4- |
| | | | | pyridyl) |
| 49 | O | phenyl | COCH₃ | CH₂(2-Cl |
| | | | | phenyl) |
| 50 | O | phenyl | COCH₃ | CH₂(3-Cl |
| | | | | phenyl) |
| 51 | O | phenyl | COCH₃ | CH₂(4-Cl |
| | | | | phenyl) |
| 52 | O | phenyl | COCH₃ | CH₂(4-CH₃ |
| | | | | phenyl) |
| 53 | O | phenyl | COCH₃ | CH₂(4-CF₃ |
| | | | | phenyl) |
| 54 | O | phenyl | COCH₃ | CH₂(4-(SO₂CH₃) |
| | | | | phenyl) |
| 55 | O | phenyl | H | CH₂-phenyl |
| 56 | O | phenyl | CH₂CH₃ | CH₂-phenyl |
| 57 | O | phenyl | SO₂CH₃ | CH₂-phenyl |
| 58 | O | phenyl | CO₂CH₃ | CH₂-phenyl |
| 59 | O | phenyl | CONHCH₂CH₃ | CH₂-phenyl |
| 60 | O | phenyl | COCH₂CH₃ | CH₂-phenyl |
| 61 | O | phenyl | CO- | CH₂-phenyl |
| | | | cyclopropyl | |
| 62 | O | phenyl | CO-phenyl | CH₂-phenyl |
| 63 | CH₂ | phenyl | COCH₃ | CH₂-phenyl |
| 64 | CH₂ | 4-F-phenyl | COCH₃ | CH₂(4-F- |
| | | | | phenyl) |

The micromolar inhibitory activity measured in vitro on KCC2 of the compounds of the invention is reported in Table 1.

TABLE 1

| | IC₅₀ |
|---|---|
| Entry | (μM) |
| 21 | 0.79 |
| 22 | 8 |
| 23 | 2.3 |
| 24 | 4 |
| 25 | 2 |
| 26 | 0.79 |
| 27 | 0.79 |
| 28 | 0.4 |
| 29 | 0.63 |
| 30 | 0.20 |
| 31 | 0.32 |
| 32 | 1 |
| 33 | 0.16 |
| 34 | 0.32 |
| 35 | 5 |
| 36 | 0.25 |
| 37 | 0.08 |
| 38 | 0.06 |
| 39 | 0.05 |
| 40 | 4 |
| 41 | 100 |
| 42 | 32 |
| 43 | >100 |
| 44 | >100 |
| 45 | 4 |
| 46 | 10 |
| 47 | 63 |
| 48 | >100 |
| 49 | 6 |
| 50 | 2.5 |
| 51 | 1.3 |
| 52 | 0.79 |
| 53 | 5 |
| 54 | >100 |
| 55 | >100 |
| 56 | >100 |
| 57 | 1.6 |
| 58 | 0.4 |
| 59 | 2 |
| 60 | 1.2 |
| 61 | 0.32 |
| 62 | 4 |
| 63 | 0.3 |
| 64 | 0.2 |
| 65 | 0.2 |
| 66 | 20 |
| 67 | 1.3 |
| 68 | 12.6 |
| 69 | 1 |

These compounds are used for the treatment of cognitive decline, preferably age-related cognitive decline.

The compounds of Formula (III), together with an adjuvant, a vehicle, a diluent or a conventionally used excipient, can be transferred in the form of pharmaceutical compositions and related unit doses, and in this form can be used as solids, for example tablets or filled capsules, or liquids, for example solutions, suspensions, emulsions, elixirs, or capsules filled with these liquids, all for oral use, or in the form of sterile injectable solutions for parenteral administration (including subcutaneous and intravenous use). These pharmaceutical compositions and related dosage forms can comprise ingredients in conventional proportions, with or without additional compounds or active ingredients, and these unit dosage forms can contain any suitable effective amount of the active ingredient proportionate to the dosage range to be employed.

The pharmaceutical compositions containing a compound of this invention can be prepared in a manner well known in the pharmaceutical art and comprise at least one active compound. In general, the compounds of this invention are administered in a pharmaceutically effective amount. The amount of the compound actually administered will by typically determined by a physician in the light of the relevant circumstances, including the condition to be treated, the administration route selected, the actual compound administered, the age, weight and response of the individual patient, the severity of the patient's symptoms, and the like.

The pharmaceutical compositions of the present invention can be administered via a variety of routes, including oral, rectal, subcutaneous, intravenous, intramuscular, intranasal and pulmonary routes.

Hereunder, the present invention will be illustrated by means of some examples, which are not intended to be considered limiting of the scope of the invention.

### Example 1. Up-Regulation of KCC2 in the Hippocampus of Elderly Humans and Mice

To verify the level of expression of KCC2, western blot analysis performed on hippocampal samples taken from elderly humans and mice, which were compared with control samples taken from young individuals.

### (1a) KCC2 is Up-Regulated in the Hippocampus of Humans Aged 75-80 Years.

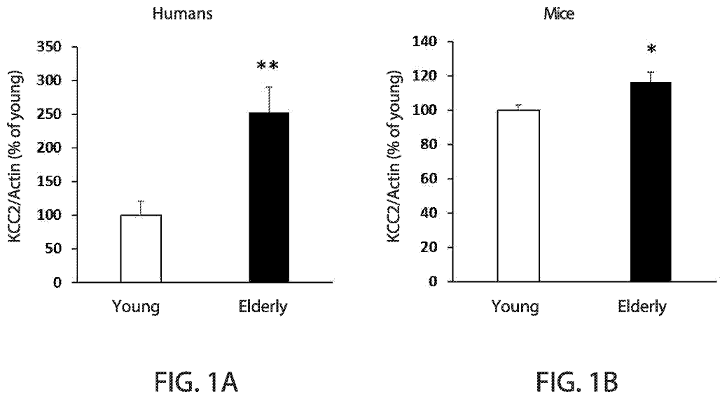

The hippocampus of humans aged 75-80 years and aged 20-25 years were obtained from the NeuroBioBank (NIH). The samples were cryo-pulverized in dry ice (−78° C.) using a stainless steel mortar. Aliquots of pulverized tissue were used for protein extraction. The protein extracts were then analysed by western blot using the following antibodies: rabbit anti-KCC2 (Millipore), rabbit anti-actin (Sigma). We found significant up-regulation of KCC2 in hippocampal samples of subjects aged 75-80 years compared to the controls aged 20-25 years (FIG. 1a).

### (1b) KCC2 is Up-Regulated in the Hippocampus of Mice Over 24 Months Old.

The hippocampi were sectioned from mice C57BL/6J over 24 months old and 2 month old control mice C57BL/6J. The protein extracts were analysed by western blot using the following antibodies: rabbit anti-KCC2 (Millipore), rabbit anti-actin (Sigma). We found significant up-regulation of KCC2 in hippocampal samples of elderly mice (>24 months old) compared to the controls (FIG. 1b).

### Example 2. Elderly Mice Exhibited Over-Sedation on Administration of Benzodiazepines

Alteration of GABAergic transmission can be reflected in an altered response to benzodiazepines.

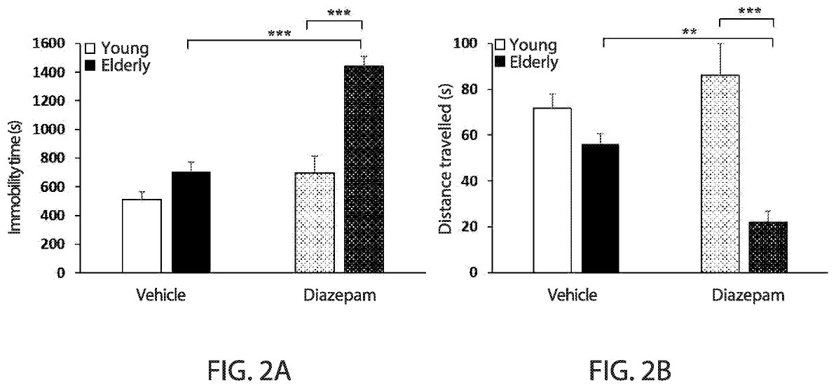

In fact, benzodiazepines are positive allosteric modulators of GABA_{A}R. Benzodiazepines are commonly used as anxiolytic drugs, but can produce anxiogenic or highly sedative effects depending on the polarity of GABAergic transmission. To test the reaction of elderly mice to benzodiazepines, mice C57BL/6J aged >24 months and 2-month old controls C57BL/6J were treated with diazepam (2 mg/kg in saline solution) or vehicle (2% DMSO in saline solution). The open field test was then carried out to evaluate locomotor activity and the level of anxiety of the mice tested. It was discovered that the elderly mice treated with diazepam exhibit a significantly higher immobility time (FIG. 2a) and a significantly lower distance travelled (FIG. 2b) compared to the elderly mice treated with the vehicle or to the young mice treated with diazepam (FIG. 2). This is indicative of an over-sedation reaction of the diazepam in the elderly mice, which may reflect the presence of hyper-inhibitory GABAergic transmission. This evidence is in accordance with the up-regulation of KCC2 (FIG. 1b).

### Example 3. The Elderly Mice Exhibited Memory Problems, which are Restored with the Inhibition of KCC2 by Compound 32

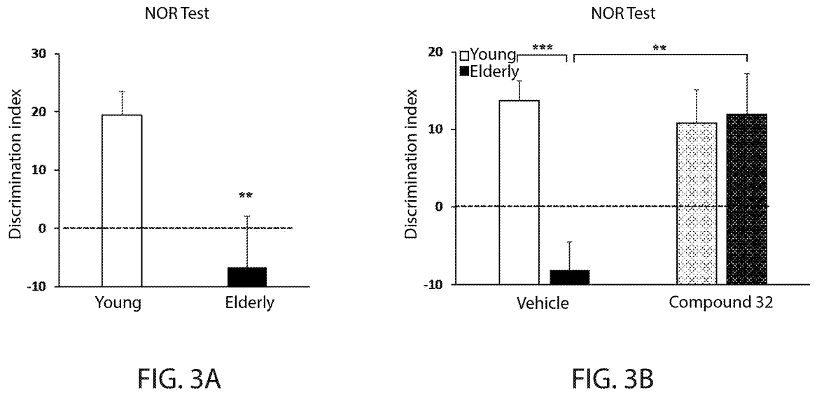

To evaluate memory capacity in the elderly mice, the novel object recognition (NOR) test was conducted. This test evaluates the long-term object recognition memory by measuring the capacity of the mice to recognize a novel object compared to familiar objects. It was discovered that the elderly mice exhibited low ability to discriminate novel objects, indicative of poor long-term memory (FIG. 3a). Subsequently, to test the ability of the non-selective KCC2 inhibitor, Compound 32, to restore poor memory, the NOR test was conducted in the presence of the drug. The mice were treated with compound 32 (1 mg/kg i.p.) or with the vehicle. Treatment with compound 32 significantly restored the poor object recognition memory of the elderly mice to the level of the young mice (FIG. 3b).

1. KCC2 inhibitors for the use in the treatment of cognitive decline, said inhibitors having formula (III): 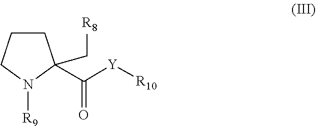
or its pharmaceutically acceptable salts, solvates and tautomers wherein:
Y is selected from the group consisting of O, CH₂ and NR₁₁ wherein R₁₁ is selected from the group consisting of H and C₁-C₄ alkyl;
R₈ is selected from the group consisting of C₁-C₄ alkyl, phenyl-C₁-C₃ alkyl, C₃-C₆ cycloalkyl, pyridyl, thienyl, and phenyl optionally substituted with at least one substituent selected from the group consisting of C₁-C₃ alkyl, C₁-C₃ alkoxyl, halogen-C₁-C₃ alkyl, halogen, methylsulphonyl, cyano;
R₉ is selected from the group consisting of H, C₁-C₄ alkyl, methylsulphonyl, C₁-C₃ alkyl-NHCO—, C₁-C₄ alkyl-CO—, phenyl-CO—, C₃-C₆ cycloalkyl-CO—, C₁-C₄ alkyl-COO;
R₁₀ is selected from the group consisting of H, C₁-C₄ alkyl, C₃-C₆ cycloalkyl-C₁-C₃ alkyl, pyridyl-C₁-C₃ alkyl, phenyl-C₁-C₃ alkyl, linear or branched, wherein the phenyl group is optionally substituted with a substituent selected from the group consisting of halogen, C₁-C₃ alkyl, halogen-C₁-C₃ alkyl, methylsulphonyl, provided that when Y is NR₁₁, R₁₀ is benzyl.

2. The KCC2 inhibitors according to claim 1, characterized in that:
Y is selected from the group consisting of O and NR₁₁ wherein R₁₁ is selected from the group consisting of H and methyl;
R₈ is selected from the group consisting of methyl, benzyl, cyclohexyl, pyridyl, thienyl and phenyl optionally substituted with at least one group selected from the group consisting of methyl, methoxyl, trifluoromethyl, fluorine, chlorine, methylsulphonyl and cyano;
R₉ is selected from the group consisting of H, ethyl, methylsulphonyl, ethylamide, CH₃—CO—, CH₃CH₂—CO—, phenyl-CO, cyclopropyl-CO, CH₃—COO—;
R₁₀ is selected from the group consisting of H, methyl, benzyl, 1-phenylethyl, 2-phenylethyl, cyclohexyl-CH₂—, pyridyl-CH₂—, chlorophenyl-CH₂—, methylphenyl-CH₂—, trifluorophenyl-CH₂—, methylsulphonylphenyl-CH₂—.

3. The KCC2 inhibitors according to claim 1, selected from the group consisting of:

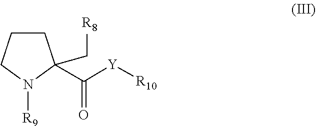

| Compound | Y | R₈ | R₉ | R₁₀ |
|---|---|---|---|---|
| 21 | O | phenyl | COCH₃ | CH₂-phenyl |
| 22 | O | CH₂-phenyl | COCH₃ | CH₂-phenyl |
| 23 | O | CH₃ | COCH₃ | CH₂-phenyl |
| 24 | O | cyclohexyl | COCH₃ | CH₂-phenyl |
| 25 | O | 4-pyridyl | COCH₃ | CH₂-phenyl |
| 26 | O | 3-thienyl | COCH₃ | CH₂-phenyl |
| 27 | O | 4-CH₃-phenyl | COCH₃ | CH₂-phenyl |
| 28 | O | 4-OCH₃-phenyl | COCH₃ | CH₂-phenyl |
| 29 | O | 4-CF₃-phenyl | COCH₃ | CH₂-phenyl |
| 30 | O | 4-F-phenyl | COCH₃ | CH₂-phenyl |
| 31 | O | 4-Cl-phenyl | COCH₃ | CH₂-phenyl |
| 32 | O | 4-(SO₂Me)- | COCH₃ | CH₂-phenyl |
| | | phenyl | | |
| 33 | O | 4-CN-phenyl | COCH₃ | CH₂-phenyl |
| 34 | O | 3-CN-phenyl | COCH₃ | CH₂-phenyl |
| 35 | O | 2-CN-phenyl | COCH₃ | CH₂-phenyl |
| 36 | O | 3-(SO₂Me)- | COCH₃ | CH₂-phenyl |
| | | phenyl | | |
| 37 | O | 3-CF₃-phenyl | COCH₃ | CH₂-phenyl |
| 38 | O | (3-CF₃, 4-F)- | COCH₃ | CH₂-phenyl |
| | | phenyl | | |
| 39 | O | (3-CF₃, 4-OCH₃)- | COCH₃ | CH₂-phenyl |
| | | phenyl h | | |
| 40 | O | (2-CF₃, 4-F)- | COCH₃ | CH₂-phenyl |
| | | phenyl | | |
| 41 | NH | phenyl | COCH₃ | CH₂-phenyl |
| 42 | NCH₃ | phenyl | COCH₃ | CH₂-phenyl |
| 43 | O | phenyl | COCH₃ | H |
| 44 | O | phenyl | COCH₃ | CH₃ |
| 45 | O | phenyl | COCH₃ | CH(CH₃) phenyl |
| 46 | O | phenyl | COCH₃ | CH₂CH₂phenyl |
| 47 | O | phenyl | COCH₃ | CH₂cyclohxyl |
| 48 | O | phenyl | COCH₃ | CH₂(4-pyridyl) |
| 49 | O | phenyl | COCH₃ | CH₂(2-Cl phenyl) |
| 50 | O | phenyl | COCH₃ | CH₂(3-Cl phenyl) |
| 51 | O | phenyl | COCH₃ | CH₂(4-Cl phenyl) |
| 52 | O | phenyl | COCH₃ | CH₂(4-CH₃ |
| | | | | phenyl) |
| 53 | O | phenyl | COCH₃ | CH₂(4-CF₃ phenyl) |
| 54 | O | phenyl | COCH₃ | CH₂(4-(SO₂CH₃) |
| | | | | phenyl) |
| 55 | O | phenyl | H | CH₂-phenyl |
| 56 | O | phenyl | CH₂CH₃ | CH₂-phenyl |
| 57 | O | phenyl | SO₂CH₃ | CH₂-phenyl |
| 58 | O | phenyl | CO₂CH₃ | CH₂-phenyl |
| 59 | O | phenyl | CONHCH₂CH₃ | CH₂-phenyl |
| 60 | O | phenyl | COCH₂CH₃ | CH₂-phenyl |
| 61 | O | phenyl | CO-cyclopropyl | CH₂-phenyl |
| 62 | O | phenyl | CO-phenyl | CH₂-phenyl |
| 63 | CH₂ | phenyl | COCH₃ | CH₂-phenyl |

and

| | | | | |
|---|---|---|---|---|
| 64 | CH₂ | 4-F-phenyl | COCH₃ | CH₂(4-F-phenyl). |

4. KCC2 inhibitors for the use in the treatment of cognitive decline, said inhibitors selected from the group consisting of:

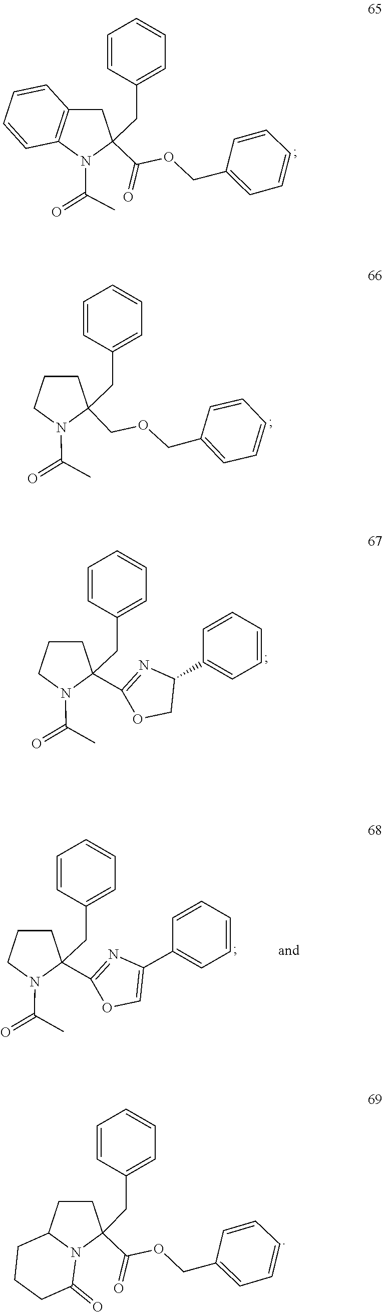 [SMILES: CC(=O)N1c2ccccc2CC1(Cc1ccccc1)C(=O)OCc1ccccc1 ; CC(=O)N1CCCC1(COCc1ccccc1)Cc1ccccc1 ; CC(=O)N1CCCC1(Cc1ccccc1)C1=N[C@H](c2ccccc2)CO1 ; CC(=O)N1CCCC1(Cc1ccccc1)c1nc(-c2ccccc2)co1 ; O=C1CCCC2CCC(Cc3ccccc3)(C(=O)OCc3ccccc3)N12]

5. The KCC2 inhibitors according to claim 1 characterized in that said cognitive decline is age-related cognitive decline.

6. The KCC2 inhibitors according to claim 4 characterized in that said cognitive decline is age-related cognitive decline.
